(19) United States
(12) Patent Application Publication
(10) Pub. No.: US 2016/0270220 A1
(43) Pub. Date: Sep. 15, 2016

(54) ELECTRONIC APPARATUS

(71) Applicant: Kabushiki Kaisha Toshiba, Tokyo (JP)

(72) Inventors: Hirofumi Omote, Adachi Tokyo (JP); Yasushi Sasaki, Mitaka Tokyo (JP); Minoru Takizawa, Sagamihara Kanagawa (JP)

(21) Appl. No.: 14/851,749

(22) Filed: Sep. 11, 2015

Related U.S. Application Data
(60) Provisional application No. 62/132,369, filed on Mar. 12, 2015.

(51) Int. Cl. H05K 1/02; A61B 5/0408; H05K 1/18; H01M 2/10; H01L 31/048

(52) U.S. Cl. CPC H05K 1/0298; H01M 2/1066; H01L 31/048; H05K 1/028; H05K 1/181; A61B 5/0408; H05K 2201/10037; H05K 2201/10113; H05K 2201/10143; H05K 2201/10121; H05K 2201/10098; H05K 2201/10151; H05K 2201/0104

(57) ABSTRACT

According to one embodiment, an electronic apparatus includes a component and a sticky gel-like portion. The gel-like portion encloses the component and has a surface, at least a portion of the surface is configured to be exposed to an outside.

### CROSS-REFERENCE TO RELATED APPLICATIONS

This application is based upon and claims the benefit of priority from U.S. Provisional Application No. 62/132,369, filed on Mar. 12, 2015; the entire contents of which are incorporated herein by reference.

### FIELD

Embodiments described herein relate generally to an electronic apparatus.

### BACKGROUND

Various electronic apparatuses may be adhered to a target face by, for example, a double-sided tape.

### BRIEF DESCRIPTION OF THE DRAWINGS

FIG. 1 is a perspective view illustrating a device, an electronic apparatus, and an external cable according to a first embodiment;

FIG. 2 is a plan view illustrating the electronic apparatus according to the first embodiment;

FIG. 3 is a cross-sectional view illustrating the electronic apparatus according to the first embodiment;

FIG. 4 is a cross-sectional view illustrating an electronic apparatus according to a second embodiment;

FIG. 5 is a plan view illustrating an electronic apparatus according to a third embodiment;

FIG. 6 is a cross-sectional view illustrating the electronic apparatus according to the third embodiment;

FIG. 7 is a plan view illustrating an electronic apparatus according to a fourth embodiment;

FIG. 8 is a cross-sectional view illustrating the electronic apparatus according to the fourth embodiment;

FIG. 9 is a plan view illustrating an electronic apparatus according to a fifth embodiment;

FIG. 10 is a cross-sectional view illustrating the electronic apparatus according to the fifth embodiment;

FIG. 11 is a plan view illustrating an electronic apparatus according to a sixth embodiment;

FIG. 12 is a cross-sectional view illustrating the electronic apparatus according to the sixth embodiment;

FIG. 13 is a plan view illustrating an electronic apparatus according to a seventh embodiment;

FIG. 14 is a cross-sectional view illustrating the electronic apparatus according to the seventh embodiment;

FIG. 15 is a plan view illustrating an electronic apparatus according to an eighth embodiment; and

FIG. 16 is a cross-sectional view illustrating the electronic apparatus according to the eighth embodiment.

### DETAILED DESCRIPTION

In general, according to one embodiment, an electronic apparatus includes a component and a sticky gel-like portion enclosing the component and having a surface, at least a portion of the surface being configured to be exposed to an outside.

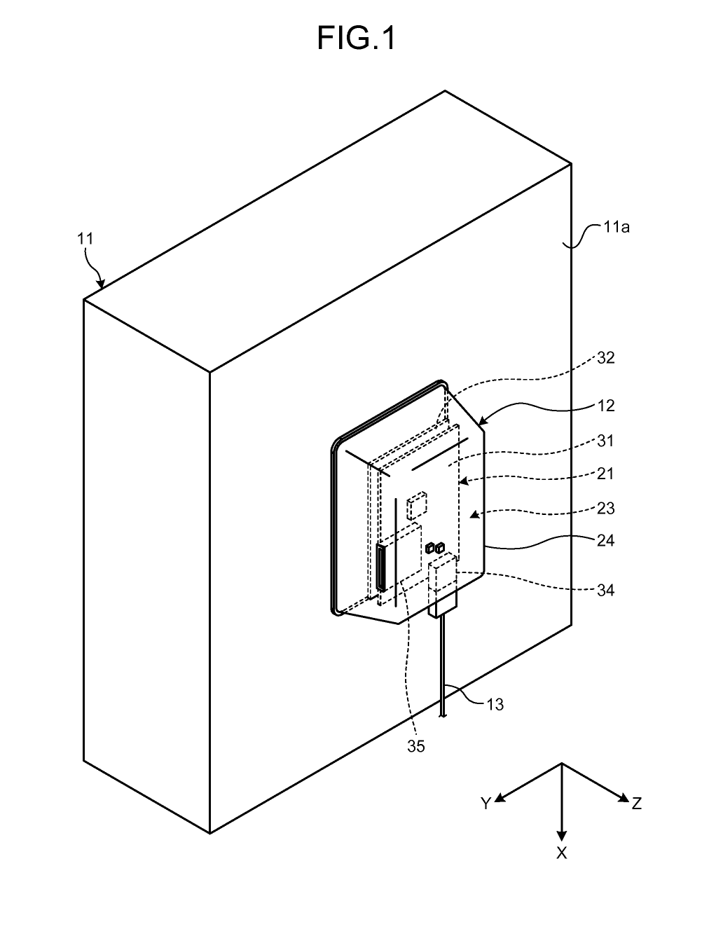

Hereinafter, a first embodiment will be described with reference to FIGS. 1 to 3. In addition, with respect to components according to the embodiments and description of the components, a plurality of expressions may be disclosed. With the components and the description thereof, other expressions which are not disclosed are not prevented. In addition, with respect to the components and the description thereof which are not disclosed together with a plurality of expressions, other expressions are not prevented.

### First Embodiment

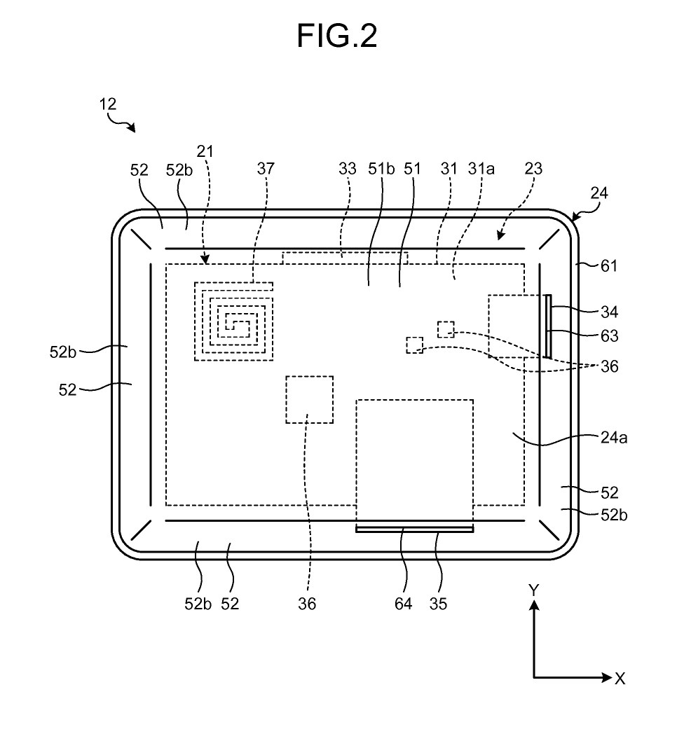

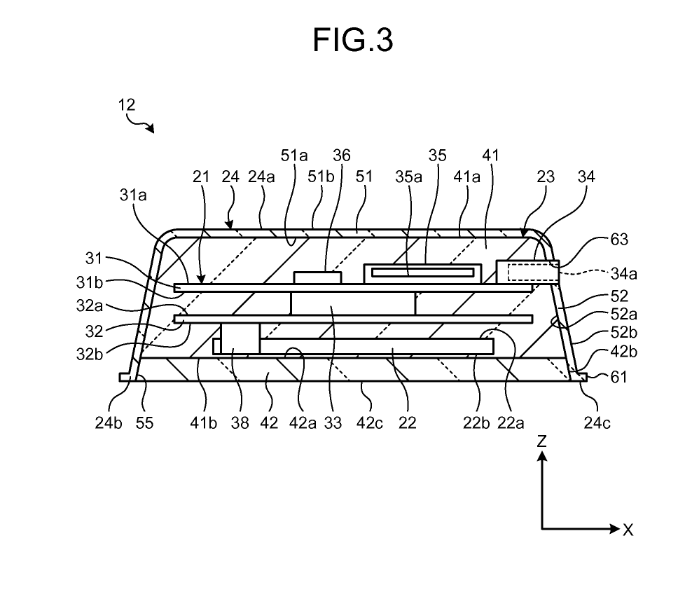

FIG. 1 is a perspective view illustrating a device 11, an electronic apparatus 12, and an external cable 13 according to a first embodiment. FIG. 2 is a plan view illustrating the electronic apparatus 12 according to the first embodiment. FIG. 3 is a cross-sectional view illustrating the electronic apparatus 12 according to the first embodiment. The electronic apparatus 12 is also referred to as, for example, an apparatus, a device, and a unit.

As illustrated in the figures, in this specification, an X axis, a Y axis, and a Z axis are defined. The X axis, the Y axis, and the Z axis are perpendicular to each other. The X axis extends along a length of the electronic apparatus 12. The Y axis extends along a width of the electronic apparatus 12. The Z axis extends along a thickness of the electronic apparatus 12.

The device 11 illustrated in FIG. 1 is, for example, a device such as a personal computer. In addition, the device 11 is not limited thereto, but the other devices may be employed. The device 11 has an outer face 11a. The outer face 11a is formed to be substantially even. In addition, the outer face 11a may be a curved face or may have unevenness.

The electronic apparatus 12 can be removably adhered to the outer face 11a of the device 11. In addition, it is not limited to the outer face 11a of the device 11, but the electronic apparatus 12 may be removably adhered to, for example, a face provided in another object such as a wall surface of a building.

As illustrated in FIG. 3, the electronic apparatus 12 includes a module 21, a battery 22, a gel-like portion 23, and a cover 24. The module 21 is an example of a component. The module 21 is also referred to as, for example, a constituent element, an element, a member, a unit, a device, and an apparatus. The gel-like portion 23 is also referred to as, for example, a gel, a jelly-like portion, a sticky portion, an adhesive portion, an elastic portion, an enclosure portion, a viscous portion, and a portion. The cover 24 is also referred to as, for example, a covering portion, an accommodating portion, a protection portion, a case, a housing, and a portion. In this application, “sticky” means, for example, mechanically, chemically, dispersively connectable between a non-adhesive element and another non-adhesive element. A sticky element can hold a heavier non-adhesive element than the sticky element at the same position as the sticky element.

The module 21 includes a first board 31, a second board 32, a third board 33, a first connector 34, a second connector 35, a plurality of electronic components 36, an antenna 37 (illustrated in FIG. 2), and a cable 38. Each of the first to third boards 31 to 33 is an example of a board. Each of the first and second connectors 34 and 35 is an example of a connector.

The first board 31 is, for example, a printed circuit board (PCB). In addition, the first board 31 may be other boards. The first board 31 has a first surface 31a and a second surface 31b.

The first surface 31a is a substantially even face facing in the direction along the Z axis. The second surface 31b is a substantially even face disposed at the opposite side of the first surface 31a. The second surface 31b faces in the direction opposite to the direction where the first surface 31a faces.

The second board 32 is, for example, a PCB. In addition, the second board 32 may be other boards. The second board 32 has a third surface 32a and a fourth surface 32b.

The third surface 32a is a substantially even surface facing in the direction along the Z axis. The fourth surface 32b is a substantially even surface disposed at the opposite side of the third surface 32a. The fourth surface 32b faces in the direction opposite to the direction where the third surface 32a faces.

The first connector 34, the second connector 35, the plurality of the electronic components 36, and the antenna 37 are mounted on the first surface 31a. In addition, the first connector 34, the second connector 35, the plurality of the electronic components 36, and the antenna 37 may be mounted on the second surface 31b, the third surface 32a, or the fourth surface 32b. Furthermore, other components may be mounted on the first to fourth surfaces 31a, 31b, 32a, and 32b.

The third surface 32a of the second board 32 faces the second surface 31b of the first board 31. The second surface 31b of the first board 31 is disposed at a position separated from the third surface 32a of the second board 32 in the direction along the Z axis.

The first board 31 and the second board 32 overlap each other in the direction along the Z axis. In other words, the first board 31 and the second board 32 overlap each other in a thickness direction of the first board 31. The thickness direction of the first board 31 is a direction where the first surface 31a on which the components such as the first connector 34, the second connector 35, the plurality of the electronic components 36, and the antenna 37 are mounted faces.

The third board 33 is, for example, a flexible printed circuit board (FPC). In addition, the third board 33 may be other boards. The third board 33 is more flexible than the first board 31 and is more flexible than the second board 32. The third board 33 is more easily elastically deformable than the first and second boards 31 and 32.

The third board 33 electrically connects the first board 31 and the second board 32. In other words, the one end portion of the third board 33 is connected to the first board 31, and the other end portion of the third board 33 is connected to the second board 32.

The third board 33 is connected to, for example, a connector mounted on the first board 31 and a connector mounted on the second board 32. The first to third boards 31 to 33 may be formed as an integrally flex-rigid printed wire board. In this case, the rigid portion is the first and the second boards 31 and 32, and the flexible portion is the third board 33.

The first connector 34 is, for example, a connector of a universal serial bass (USB). In addition, the first connector 34 may be, for example, other connectors such as a connector of a local area network (LAN) and a connector of High definition multimedia interface (HDMI).

An insert opening 34a is provided in the first connector 34. The insert opening 34a is an example of a second opening. For example, a plug of the external cable 13 of FIG. 1 is inserted into the insert opening 34a. The external cable 13 is, for example, a USB cable which is connected to the device 11. Therefore, the first connector 34 is connected through the external cable 13 to the device.

The second connector 35 is, for example, a connector of an SD card. In addition, the second connector 35 is not limited thereto, but the other connectors such as a connector of a micro SD card or a connector of a SIM card may be employed.

A slot 35a is provided in the second connector 35. The slot 35a is an example of a second opening. For example, an SD card is inserted into the slot 35a. Therefore, the SD card is connected to the second connector 35.

The antenna 37 illustrated in FIG. 2 is, for example, an antenna for a wireless LAN. In addition, the antenna 37 is not limited thereto, but other antennas such as an antenna for a third generation (3G) mobile communication system or an antenna for Bluetooth (registered trade mark) may be employed. Due to the antenna 37, the module 21 can, for example, radio-communicate with other devices.

The battery 22 illustrated in FIG. 3 is, for example, a lithium ion secondary battery. In addition, the battery 22 may be another secondary battery such as a nickel metal hydride rechargeable battery or a primary battery. The battery 22 is formed in a plate shape. In addition, the battery 22 may be formed in another shape such as a cylindrical shape.

The battery 22 has a fifth surface 22a and a sixth surface 22b. The fifth surface 22a is a substantially even surface facing in the direction along the Z axis. The sixth surface 22b is a substantially even surface disposed at the opposite side of the fifth surface 22a. The sixth surface 22b faces in the direction opposite to the direction where the fifth surface 22a faces.

The fifth surface 22a of the battery 22 faces the fourth surface 32b of the second board 32. The fourth surface 32b of the second board 32 is disposed at a position separated from the fifth surface 22a of the battery 22 in the direction along the Z axis. The second board 32 and the battery 22 overlap each other in the direction along the Z axis. In other words, the second board 32 and the battery 22 overlap each other in the thickness direction of the first board 31.

The battery 22 is connected to the second board 32 by a flexible cable 38. Therefore, the battery 22 supplies electric power to the module 21. The battery 22 may be charged with electric power supplied from an outside through the external cable 13.

The gel-like portion 23 is flexible and keeps a given form, and made of a gel that is a semi-solid colloidal suspension of a solid dispersed in a liquid. The gel-like portion 23 may be made of alternative materials or gel. The gel-like portion 23 includes a protection portion 41 and an adhesive portion 42. The protection portion 41 according to this embodiment is an example of a second gel-like member. The protection portion 41 is also referred to as, for example, an enclosure portion, a covering portion, and a retaining portion. The adhesive portion 42 is an example of a first gel-like member. The adhesive portion 42 is also referred to as, for example, a sticky portion, an attaching portion, and a viscous portion.

The protection portion 41 is a sticky gel-like member. The protection portion 41 is made of, for example, an insulating silicon gel. Therefore, the protection portion 41 is softer than the first board 31. In addition, the protection portion 41 is not limited thereto, but the protection portion may be made of other materials.

The protection portion 41 encloses the module 21 and the battery 22. In other words, the protection portion 41 covers the module 21. Each of the first board 31, the second board 32, the third board 33, the electronic components 36, the antenna 37, and the cable 38 is enclosed in the protection portion 41. Each of the first connector 34 and the second connector 35 is disposed in the protection portion 41. The protection portion 41 according to this embodiment exposes a portion of the first connector 34 and a portion of the second connector 35 to an outside and covers other portions of the module 21. In addition, the protection portion 41 is not limited thereto, but the protection portion may expose other portions of the module 21 or may completely enclose the module 21. In this application, “enclose” means covering a target object from upper, lower, right, left, front, and back sides viewed from the target object.

A portion of the protection portion 41 is interposed between the second surface 31b of the first board 31 and the third surface 32a of the second board 32. Therefore, the protection portion 41 maintains the first board 31 to be at a position separated from the second board 32.

A portion of the protection portion 41 is interposed between the fourth surface 32b of the second board 32 and the fifth surface 22a of the battery 22. Therefore, the protection portion 41 maintains the second board 32 to be at a position separated from the battery 22.

The protection portion 41 is attached to the respective surfaces of the battery 22, the first board 31, the second board 32, the third board 33, the first connector 34, the second connector 35, the electronic components 36, the antenna 37, and the cable 38. In addition, gaps may be formed between the protection portion 41 and the respective surfaces of the battery 22, the first board 31, the second board 32, the third board 33, the first connector 34, the second connector 35, the electronic components 36, the antenna 37, and the cable 38.

The protection portion 41 has a first adhesive face 41a and a second adhesive face 41b. The first adhesive face 41a is an outer face of the protection portion 41 facing the cover 24. The second adhesive face 41b is an outer face of the protection portion 41 facing the adhesive portion 42.

The adhesive portion 42 is a sticky gel-like portion. The adhesive portion 42 is made of, for example, an insulating silicon gel. Therefore, the adhesive portion 42 is softer than the first board 31. In addition, the adhesive portion 42 is not limited thereto, but adhesive portion 42 may be made of other materials.

The adhesive portion 42 has a third adhesive face 42a, a fourth adhesive face 42b, and a fifth adhesive face 42c. The fifth adhesive face 42c is an example of a first face and is also referred to as, for example, a sticky portion, an attaching portion, and a viscous portion.

The third adhesive face 42a is an outer face of the adhesive portion 42 facing the second adhesive face 41b of the protection portion 41. The fourth adhesive face 42b is an outer face of the adhesive portion 42 facing the cover 24. The fifth adhesive face 42c is an outer face of the adhesive portion 42 disposed at the opposite side of the third adhesive face 42a. The fifth adhesive face 42c is an outer face of the gel-like portion 23. The adhesive portion 42 is disposed between the fifth adhesive face 42c and the protection portion 41 in the direction along the Z-axis.

The fifth adhesive face 42c faces in the direction along the Z axis. The direction where the fifth adhesive face 42c faces is the same as the direction where the second surface 31b of the first board 31 faces. In addition, the fifth adhesive face 42c may face in another direction.

The fifth adhesive face 42c is formed to be substantially even. In addition, the fifth adhesive face 42c may be swollen in a convex shape or may be recessed in a concave shape. Since the adhesive portion 42 is a gel-like portion, the shape of the fifth adhesive face 42c is easily deformed by, for example, gravity.

The silicon gel which is a material of the protection portion 41 and the adhesive portion 42 is removably adhered to other objects which are in contact with the outer face of the silicon gel. An adhesive force of the silicon gel which is a material of the protection portion 41 and the adhesive portion 42 is, for example, decreased by wetting with water and recovered by drying. In addition, properties of the silicon gel which is a material of the protection portion 41 and the adhesive portion 42 are not limited thereto.

The second adhesive face 41b of the protection portion 41 is in contact with the third adhesive face 42a of the adhesive portion 42. Therefore, the second adhesive face 41b of the protection portion 41 is adhered to the third adhesive face 42a of the adhesive portion 42. The adhesive force between the protection portion 41 and the adhesive portion 42 is stronger than the adhesive force between the protection portion 41 and the other objects and is stronger than the adhesive force between the adhesive portion 42 and the other objects. In addition, the adhesive force between the protection portion 41 and the adhesive portion 42 is not limited thereto.

The protection portion 41 is made of a silicon gel which is added with a reinforcement material. The reinforcement material is, for example, fiber or a synthetic rubber. The adhesive portion 42 is made of the above-described silicon gel having no reinforcement material. Therefore, the protection portion 41 is harder than the adhesive portion 42. In addition, the silicon gel which is a material of the adhesive portion 42 may be added with a reinforcement material.

The silicon gel which is a material of the protection portion 41 and the adhesive portion 42 is, for example, transparent (translucent). In addition, the silicon gel which is a material of the protection portion 41 and the adhesive portion 42 may be colored entirely or partially.

The cover 24 is a flexible member formed in a substantially frustum-shaped box form of which one plane is opened. The cover 24 is made of, for example, a silicon rubber. In addition, the shape and material of the cover 24 are not limited thereto.

The cover 24 is softer than the first board 31. The cover 24 is harder than the gel-like portion 23. Therefore, the cover 24 can maintain the gel-like portion 23 to be in a certain shape. The hardness of the cover 24 is set according to, for example, a thickness of the cover 24 and a hardness of the material. The cover 24 has a first wall 51 and a plurality of second walls 52. The second wall 52 is an example of a wall and is also referred to as, for example, a member and a portion.

The first wall 51 is formed, for example, in a substantially quadrangular plate shape. In addition, the first wall 51 is not limited thereto, but the first wall 51 may be formed in other shapes. The first wall 51 has a first inner face 51a and a first outer face 51b.

The first inner face 51a is a substantially even face facing in the direction along the Z axis. The first inner face 51a faces toward the inside of the electronic apparatus 12 and faces the first adhesive face 41a of the protection portion 41. The first outer face 51b is a substantially even face disposed at the opposite side of the first inner face 51a. The first outer face 51b faces in the direction opposite to the first inner face 51a.

The first inner face 51a of the first wall 51 faces the first surface 31a of the first board 31 through the protection portion 41. The first surface 31a of the first board 31 is disposed at a position separated from the first inner face 51a of the first wall 51 in the direction along the Z axis. The first board 31 and the first wall 51 overlap each other in the direction along the Z axis. In other words, the first board 31 and the first wall 51 overlap each other in the thickness direction of the first board 31.

A portion of the protection portion 41 is interposed between the first surface 31a of the first board 31 and the first inner face 51a of the first wall 51. Therefore, the protection portion 41 maintains the first board 31 to be at a position separated from the first wall 51.

The second wall 52 is formed, for example, in a substantially quadrangular plate shape. In addition, the second wall 52 is not limited thereto, but the wall may be formed in other shapes. Each of the second walls 52 extends from the edge of the first wall 51 in a direction obliquely intersecting the fifth adhesive face 42c. The direction obliquely intersecting the fifth adhesive face 42c is an example of a second direction. The second wall 52 may extend in other directions such as a direction perpendicular to the fifth adhesive face 42c.

Each of the second walls 52 has a second inner face 52a and the second outer face 52b. The second inner face 52a faces toward the inside of the electronic apparatus 12 and faces the first adhesive face 41a of the protection portion 41 and the fourth adhesive face 42b of the adhesive portion 42. The second outer face 52b is a substantially even surface disposed at the opposite side of the second inner face 52a. The second outer face 52b faces in the direction opposite to the second inner face 52a.

The cover 24 further includes a first end portion 24a and a second end portion 24b in the direction along the Z axis. The second end portion 24b is an example of an end. The first end portion 24a is closed by the first wall 51. In other words, the first wall 51 forms the first end portion 24a of the cover 24. The second end portion 24b is disposed at the opposite side of the first end portion 24a.

A first opening portion 55 is opened in the second end portion 24b of the cover 24. The first opening portion 55 is an example of a first opening and is also referred to as, for example, an opened portion, an exposure portion, and a portion. In other words, the second end portion 24b is an opened portion of a box-shaped cover.

The first opening portion 55 is defined by the first inner face 51a of the first wall 51 and the second inner face 52a of the second wall 52. In addition, the first opening portion 55 is not limited thereto. The one end portion of the second wall 52 in the direction along the Z axis is connected to the edge of the first wall 51. The other end portion of the second wall 52 in the direction along the Z axis forms the second end portion 24b of the cover 24. Therefore, the opened portion of the first opening portion 55 is formed by the other end portion of the second wall 52.

The gel-like portion 23 enclosing the module 21 is accommodated in the first opening portion 55 of the cover 24. The gel-like portion 23 is accommodated in the cover 24, so that the first adhesive face 41a of the protection portion 41 is adhered to the first inner face 51a. Furthermore, the first adhesive face 41a of the protection portion 41 and the fourth adhesive face 42b of the adhesive portion 42 are adhered to the second inner face 52a. In this application, “accommodated” means being inside of a target object. When a first element is accommodated in a second element, the first element may exposed from at least one side of the second element.

The fifth adhesive face 42c of the adhesive portion 42 is exposed by the first opening portion 55. In other words, a portion (fifth adhesive face 42c) of the gel-like portion 23 which is exposed by the first opening portion 55 is formed by the adhesive portion 42. The adhesive portion 42 is accommodated in the cover 24 and covers the protection portion 41 which is accommodated in the same cover 24. In addition, a portion of the protection portion 41 may be exposed by the first opening portion 55.

The cover 24 further includes a flange portion 61. The flange portion 61 is an example of a protrusion and is also referred to as, for example, a convex portion, a protruding portion, a waterproof portion, a contact portion, and a portion. The flange portion 61 is provided in the second end portion 24b of the cover 24. In other words, the flange portion 61 is provided on the second wall 52.

The flange portion 61 protrudes from the second outer face 52b of the second wall 52 toward the outside of the first opening portion 55 in the second end portion 24b of the cover 24. The flange portion 61 protrudes in the direction along the fifth adhesive face 42c of the adhesive portion 42 which is exposed by the first opening portion 55.

The end surface 24c of the cover 24 in the direction along the Z axis is formed by the flange portion 61 and the second end portion 24b of the cover 24. The second end portion 24b is a portion of the cover 24 including the end surface 24c. The end surface 24c forms substantially the same plane as the fifth adhesive face 42c of the adhesive portion 42 which is exposed by the first opening portion 55. In addition, for example, the fifth adhesive face 42c may be provided at a position protruding from the end surface 24c or may be provided at a position recessed from the end surface 24c.

As illustrated in FIG. 2, two second opening portions 63 and 64 are further provided in the cover 24. Each of the second opening portions 63 and 64 is an example of a third opening and also referred to as, for example, an opened portion, an exposure portion, and a portion. Each of the second opening portions 63 and 64 is provided on, for example, the second wall 52. In addition, the second opening portions 63 and 64 may be provided on the first wall 51.

The second opening portion 63 is provided at the position corresponding to the first connector 34 and has the size corresponding to the first connector 34. The first connector 34 passes through the second opening portion 63. The edge of the cover 24 defining the second opening portion 63 is in elastic contact with the outer surface of the first connector 34. A gap between the second opening portion 63 and the first connector 34 may be closed by, for example, a synthetic resin.

The insert opening 34a of the first connector 34 is exposed to the outside of the electronic apparatus 12 by the second opening portion 63. Therefore, for example, a plug of the external cable 13 can be inserted from the outside of the electronic apparatus 12 into the insert opening 34a of the first connector 34.

The second opening portion 64 is provided at the position corresponding to the second connector 35 and has the size corresponding to the second connector 35. The second connector 35 passes through the second opening portion 64. The edge of the cover 24 defining the second opening portion 64 is in elastic contact with the outer surface of the second connector 35. A gap between the second opening portion 64 and the second connector 35 may be closed by, for example, a synthetic resin.

The slot 35a of the second connector 35 is exposed to the outside of the electronic apparatus 12 by the second opening portion 64. Therefore, for example, an SD card can be inserted from the outside of the electronic apparatus 12 into the slot 35a of the second connector 35.

The electronic apparatus 12 formed in the above-described manner is used as, for example, peripheral apparatuses of the device 11 as described hereinafter. In addition, the electronic apparatus 12 is not limited to the use described below, but the electronic apparatus may be used for the other uses.

For example, a synthetic resin film is adhered to the fifth adhesive face 42c of the adhesive portion 42 which is exposed by the first opening portion 55 in advance. Therefore, adhesion of moisture or dust to the fifth adhesive face 42c is suppressed.

For example, in the case where the electronic apparatus 12 is to be attached to the outer face 11a of the device 11, the film is peeled off, so that the fifth adhesive face 42c is exposed. The fifth adhesive face 42c is brought into contact with the outer face 11a of the device 11. Therefore, the fifth adhesive face 42c of the adhesive portion 42 which is a sticky gel-like portion is adhered to the outer face 11a of the device 11. In other words, the electronic apparatus 12 is attached to the outer face 11a of the device 11.

If the fifth adhesive face 42c is adhered to the outer face 11a of the device 11, the end surface 24c of the cover 24 is in contact with the outer face 11a. Therefore, the flange portion 61 is in the state that the flange portion 61 extends along the outer face 11a, so that water permeation through the gap between the outer face 11a and the end surface 24c is suppressed. In addition, the flange portion 61 may be covered with packing or gel, so that water permeation through the gap between the outer face 11a and the end surface 24c may be suppressed.

For example, a plug of the external cable 13 is inserted into the insert opening 34a of the first connector 34 which is exposed by the second opening portion 63. The device 11 and the electronic apparatus 12 are electrically connected to each other by the external cable 13. Therefore, the device 11 can use the second connector 35 and the antenna 37 of the electronic apparatus 12. In addition, it is not limited to the external cable 13, but the electronic apparatus 12 may be wirelessly connected to the device 11 by using the antenna 37.

The battery 22 supplies power to the module 21. Therefore, the electronic apparatus 12 can operate for a relatively long time without supplying electric power from an outside. The battery 22 may be charged by the electric power from an external power supply such as a device 11 through the external cable 13.

The fifth adhesive face 42c of the electronic apparatus 12 can be peeled off from the outer face 11a of the device 11. The fifth adhesive face 42c peeled off from the outer face 11a continues to have stickiness. Therefore, the fifth adhesive face 42c can be adhered to a target face such as the outer face 11a again. In the case where the adhesive force of the fifth adhesive face 42c is decreased, the fifth adhesive face 42c recovers the adhesive force, for example, by cleaning with water and detergent and drying. In addition, the fifth adhesive face 42c may be fixed on a target face, for example, by thermal curing of the gel-like portion 23 or by being cured with another solution.

The above-described electronic apparatus 12 is manufactured, for example, as described hereinafter. In addition, it is not limited to the method described hereinafter, but the electronic apparatus 12 may be manufactured by the other methods.

First, the cover 24 is made of, for example, a silicon rubber. The cover 24 is manufactured by, for example, injection molding. In addition, the cover 24 may be manufactured by other methods.

Next, the module 21 is accommodated in the first opening portion 55 of the cover 24. The module 21 is supported at a position separated from the cover 24, for example, by a jig. Furthermore, the second board 31, the second board 32, and the battery 22 are supported at positions separated from each other, for example, by a jig. The first and second connectors 34 and 35 of the module 21 pass through the second opening portions 63 and 64 of the cover 24.

Next, a material of the protection portion 41 is supplied to the first opening portion 55 of the cover 24. In addition, during the material supplying period, the material of the protection portion 41 has fluidity. This material covers the module 21. In other words, the module 21 is buried in the material. In addition, a portion of the module 21 may be exposed.

Next, the jig supporting the module 21 is removed, and bubbles in the material are removed, for example, by a degassing process. Furthermore, the material having fluidity is hardened by heating. Therefore, the protection portion 41 is formed.

For example, the electronic apparatus 12 which is supplied with the material having fluidity is accommodated in a chamber, and the chamber is decreased in pressure for a predetermined time, for example, by a vacuum pump, so that the degassing process is performed. Therefore, air contained in the material during the material supplying period is extracted and removed from the material, so that, due to the heating, generating bubbles by the air in the material is suppressed.

The first adhesive face 41a of the protection portion 41 is hardened to be adhered to the first inner face 51a of the first wall 51 and the second inner face 52a of the second wall 52. Since the protection portion 41 is a sticky gel-like portion, peeling off the protection portion 41 from the first and second walls 51 and 52 is suppressed.

Next, the material of the adhesive portion 42 is supplied to the first opening portion 55 of the cover 24. In addition, during the material supplying period, the material of the adhesive portion 42 has fluidity. The material covers the protection portion 41. In addition, a portion of the protection portion 41 may be exposed.

Next, bubbles in the material are removed, for example, by a degassing process. Furthermore, the material having fluidity is hardened by heating. Therefore, the adhesive portion 42 is formed. The fifth adhesive face 42c of the formed adhesive portion 42 is exposed by the first opening portion 55 of the cover 24.

The fourth adhesive face 42b of the adhesive portion 42 is hardened to be adhered to the second adhesive face 41b of the protection portion 41 and the second inner face 52a of the second wall 52. Since the adhesive portion 42 is a sticky gel-like portion, peeling off the adhesive portion 42 from the protection portion 41 and the second wall 52 is suppressed.

According to the processes described above, the electronic apparatus 12 is manufactured. In addition, the material of the protection portion 41 and the material of the adhesive portion 42 may be simultaneously heated. In addition, after the protection portion 41 and the adhesive portion 42 are formed, the second opening portions 63 and 64 may be formed.

In the electronic apparatus 12 according to the first embodiment, the module 21 is enclosed in the sticky gel-like portion 23, and the gel-like portion 23 has the fifth adhesive face 42c which is exposed by the first opening portion 55 of the cover 24. Therefore, if the fifth adhesive face 42c is brought into contact with a target face such as the outer face 11a of the device 11, the electronic apparatus 12 is removably adhered to the surface. Therefore, for example, without other members such as a double-sided tape, the electronic apparatus 12 including the module 21 can be adhered to the target face. Furthermore, the gel-like portion 23 encloses the module 21, so that exposing the module 21 to, for example, impact, a chemical agent, wind and rain, and a corrosion gas is suppressed. Therefore, it is possible to improve durability of the module 21. Furthermore, the cover 24 accommodates the gel-like portion 23, and thus, the electronic apparatus 12 is adhered to the target face, so that adherence of the gel-like portion 23 to user's fingers is suppressed. Furthermore, the gel-like portion 23 absorbs noise generated by the module 21, so that emitting the noise to the outside is suppressed.

The battery 22 supplying electric power to the module 21 is enclosed in the gel-like portion 23. Therefore, the electronic apparatus 12 can operate by the electric power of the battery 22, and the usage and available period of the electronic apparatus 12 are extended. Furthermore, the electric power of the battery 22 may be supplied to other devices such as the device 11. Furthermore, the gel-like portion 23 encloses the battery 22, so that exposing the battery 22 to, for example, impact, a chemical agent, wind and rain, and a corrosion gas is suppressed.

The gel-like portion 23 has the adhesive portion 42 having the fifth adhesive face 42c and the protection portion 41 enclosing the module 21, and the protection portion 41 is harder than the adhesive portion 42. Therefore, the hardness of the protection portion 41 protecting the module 21 is improved. Therefore, even in the case where the protection portion 41 is thinned, the module 21 can be protected by the gel-like portion 23, so that it is possible to miniaturize the electronic apparatus 12. Furthermore, since the adhesive portion 42 is added with no reinforcement material, the adhesive portion 42 can be more strongly adhered to the target face than to the protection portion 41.

The module 21 includes the antenna 37. Therefore, for example, various devices such as the device 11 can wirelessly communicate through the antenna 37 of the electronic apparatus 12. Since the electronic apparatus 12 can be adhered to the target face, it is possible to easily attach the antenna 37 at a desired site.

The module 21 includes the first and second boards 31 and 32 and the flexible third board 33 connecting the first and second boards 31 and 32. Therefore, the area of a portion where, for example, electronic components such as the first connector 34, the second connector 35, the electronic components 36, and the antenna 37 can be mounted is increased. Furthermore, even in the case where an external force is exerted to the first and second boards 31 and 32 through the cover 24 and the gel-like portion 23, the external force is absorbed by the soft third board 33, so that damage to the first and second boards 31 and 32 is suppressed.

The first and second boards 31 and 32 overlap in the thickness direction of the first board 31. Therefore, at least one of the length of the electronic apparatus 12 in the direction along the X axis and the length in the direction along the Y axis can be reduced. Furthermore, the length of the electronic apparatus 12 in the direction along the Z axis is increased, so that the user easily carries the electronic apparatus 12.

The cover 24 has a flange portion 61 which protrudes in the direction along the fifth adhesive face 42c toward the outside of the first opening portion 55 from the second end portion 24b where the first opening portion 55 is opened. Therefore, when the fifth adhesive face 42c exposed by the first opening portion 55 is in contact with the target face, the flange portion 61 extends along the target face. Therefore, for example, it is suppressed that a liquid such as water permeates the fifth adhesive face 42c from the gap between the second end portion 24b of the cover 24 and the target face.

The module 21 includes the first connector 34 provided with the insert opening 34a exposed by the second opening portion 63 of the cover 24 and the second connector 35 provided with the slot 35a exposed by the second opening portion 64. Therefore, the module 21 is in the state that the module is enclosed in the gel-like portion 23, so that the module 21 can be connected to an external device such as the external cable 13 or an SD card through the first and second connectors 34 and 35. Since the electronic apparatus 12 can be easily adhered to the target face, it is possible to install the first and second connectors 34 and 35 at desired sites.

In the first embodiment, the gel-like portion 23 has the protection portion 41 made of a silicon gel added with a reinforcement material and the adhesive portion 42 made of a silicon gel. However, the gel-like portion 23 is not limited thereto, but the gel-like portion 23 may be made of one silicon gel, enclose the module 21, and form a face (fifth adhesive face 42c) which can be adhered to the target face such as the outer face 11a of the device 11. Namely, the protection portion 41 and the adhesive portion 42 may be made of the same material generally. In addition, the gel-like portion 23 may have three or more portions.

### Second Embodiment

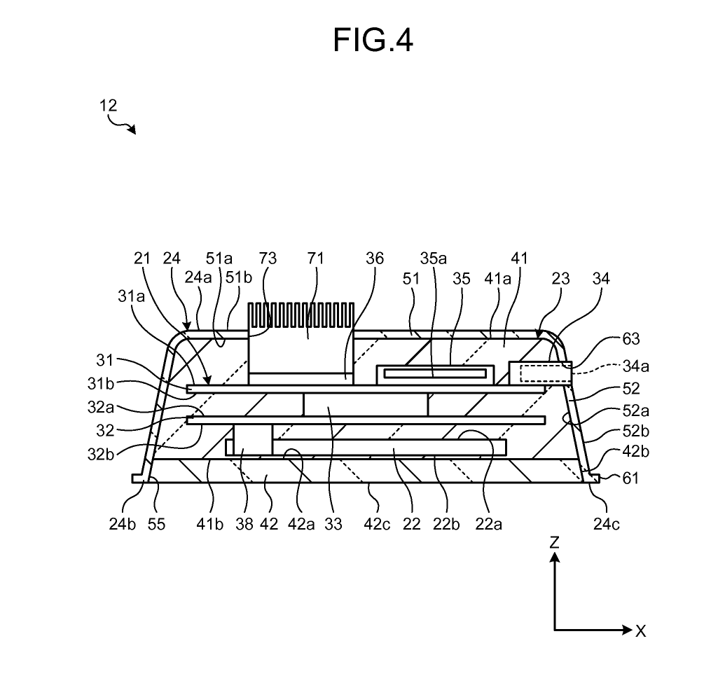

Hereinafter, a second embodiment will be described with reference to FIG. 4. In addition, in the description of the plural embodiments hereinafter, the components having the same functions as those of the above-described components are denoted by the same reference numerals denoting the above-described components, and in some cases, the further description thereof may be omitted. In addition, the plural components denoted by the same reference numerals do not necessarily have the same functions and properties, but the components may have different functions and properties according to the embodiments.

FIG. 4 is a cross-sectional view illustrating an electronic apparatus 12 according to the second embodiment. The module 21 according to the second embodiment includes a heat sink 71. The heat sink 71 is also referred to as, for example, a heat radiating portion and a member.

The heat sink 71 is made of, for example, a material such as an aluminum alloy. The heat sink 71 is installed in, for example, an electronic component 36 radiating heat such as a micro-processing unit (MPU). The heat sink 71 extends from the installed electronic components 36 toward the first wall 51 of the cover 24.

A third opening portion 73 is provided on the first wall 51 of the cover 24. The third opening portion 73 is provided at the position corresponding to the heat sink 71 and has the size corresponding to the heat sink 71. A portion of the heat sink 71 passes through the third opening portion 73. In other words, the heat sink 71 is exposed to the outside of the electronic apparatus 12 by the third opening portion 73.

The edge of the cover 24 defining the third opening portion 73 is in elastic contact with the outer face of the heat sink 71. A gap between the third opening portion 73 and the heat sink 71 may be closed by, for example, a synthetic resin.

The protection portion 41 according to the second embodiment is made of a silicon gel which is added with a conductive material and a thermal conductive material as well as a reinforcement material. The conductive material is, for example, a metal powder or metal filler. The thermal conductive material is, for example, metal filler or a synthetic resin. The thermal conductivity of the thermal conductive material is higher than the thermal conductivity of the silicon gel.

Since the conductive material is added, the protection portion 41 has conductivity. The adhesive portion 42 has insulating properties as described above. Furthermore, since the thermal conductive material is added, the thermal conductivity of the protection portion 41 is higher than the thermal conductivity of the adhesive portion 42.

The conductive portion of the module 21 enclosed in the protection portion 41 described above is covered with, for example, an insulating body. Therefore, occurring short-circuit between the conductive portion of the module 21 and the protection portion 41 is suppressed.

In the electronic apparatus 12 according to the second embodiment, the gel-like portion 23 has a conductive protection portion 41 enclosing the module 21. Therefore, with respect to the module 21, the module 21 can be electronically shielded by the conductive protection portion 41 without an electromagnetic shield member. Therefore, the electronic apparatus 12 becomes lightweight. For example, due to the weight of the electronic apparatus 12, naturally peeling off the electronic apparatus 12 from the target face is suppressed. In addition, the module 21 may be covered with the electromagnetic shield member such as a metal case.

The gel-like portion 23 has the protection portion 41 which encloses the module 21 and has thermal conductivity higher than that of the adhesive portion 42. Therefore, heat is radiated from the module 21 enclosed in the gel-like portion 23 by the protection portion 41.

The heat sink 71 is thermally connected to the heat radiating electronic component 36. The heat sink 71 is exposed to the outside by the third opening portion 73. Therefore, the electronic components 36 are more efficiently cooled. Furthermore, a portion of the heat sink 71 is disposed in the protection portion 41 added with the thermal conductive material. Therefore, the module 21 is more efficiently cooled by the heat sink 71.

In the second embodiment, the heat sink 71 passes through the third opening portion 73 provided on the first wall 51. However, the heat sink 71 is not limited thereto, but for example, the heat sink may pass through an opening provided on the second wall 52. Namely, the direction where the heat sink 71 protrudes to the outside of the electronic apparatus 12 is not limited. The electronic apparatus 12 can be adhered to the target face such as the outer face 11a of the device 11 in various directions. Therefore, the electronic apparatus 12 may be adhered to the outer face 11a, for example, in the direction where it can be suppressed that the heat sink 71 interferes with the device 11.

In the second embodiment described above, the conductive material and the thermal conductive material are added to the silicon gel which is a material of the protection portion 41. However, the material added to the material of the protection portion 41 is not limited thereto, but for example, a magnetic material filler may be added. Therefore, the module 21 is magnetically shielded. Therefore, for example, in the case where the module 21 is a microphone or speaker having a permanent magnet, the permanent magnet is shielded, so that it is suppressed that the electronic component such as a magnetoresistive random access memory (MRAM) provided in the device 11 is influenced by a magnetic field of the permanent magnet. Similarly, for example, in the case where the device 11 has a permanent magnet and an electronic component such as an MRAM is mounted in the module 21, it is suppressed that the electronic component is influenced by an external magnetic field such as a magnetic field of the permanent magnet of the device 11.

### Third Embodiment

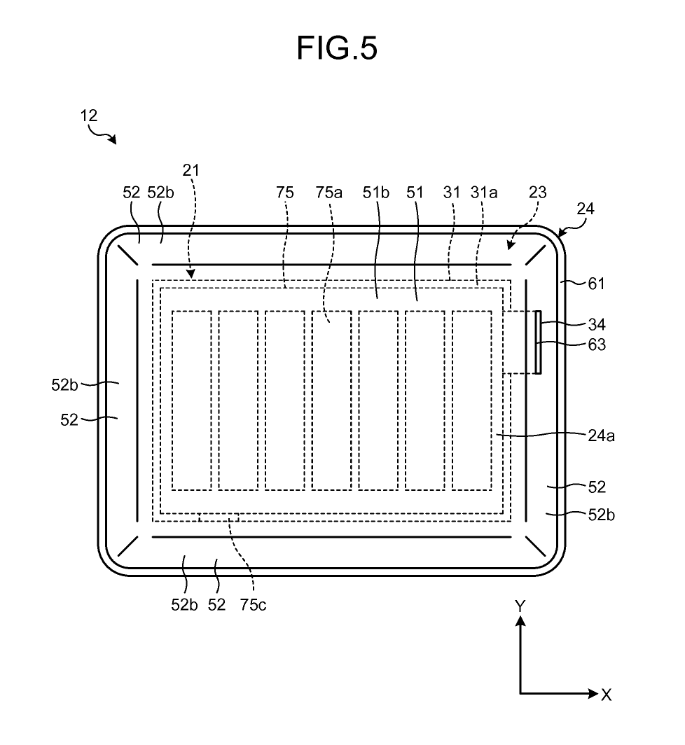

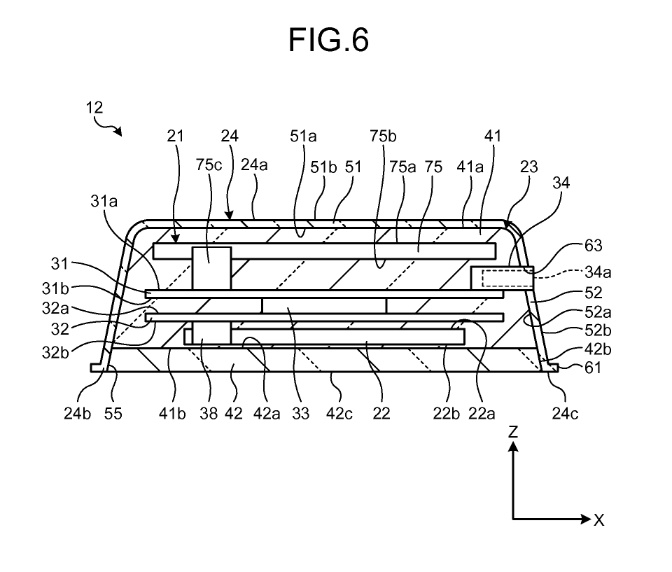

Hereinafter, a third embodiment will be described with reference to FIGS. 5 and 6. FIG. 5 is a plan view illustrating an electronic apparatus 12 according to the third embodiment. FIG. 6 is a cross-sectional view illustrating the electronic apparatus 12 according to the third embodiment.

As illustrated in FIG. 6, the module 21 according to the third embodiment further includes a solar cell 75. The solar cell 75 is also referred to as, for example, a photocell, an electric power apparatus, and a power generating part. The solar cell 75 is formed in a substantially quadrangular plate shape.

The solar cell 75 is enclosed in the protection portion 41 of the gel-like portion 23. The solar cell 75 has a light-receiving face 75a, a back face 75b, and a feeding cable 75c. The light-receiving face 75a is an example of a second face. The light-receiving face 75a is a substantially even surface facing in the direction along the Z axis. The back face 75b is a substantially even surface disposed at the opposite side of the light-receiving face 75a. The back face 75b faces in the direction opposite to the direction where the light-receiving face 75a faces.

The light-receiving face 75a of the solar cell 75 faces the first inner face 51a of the first wall 51 through the protection portion 41. The light-receiving face 75a of the solar cell 75 is disposed at a position separated from the first inner face 51a of the first wall 51 in the direction along the Z axis. The first wall 51 and the solar cell 75 overlap each other in the direction along the Z axis. In other words, the solar cell 75 overlaps with the first wall 51 in the thickness direction of the first wall 51.

The back face 75b of the solar cell 75 faces the first surface 31a of the first board 31 through the protection portion 41. The back face 75b of the solar cell 75 is disposed at a position separated from the first surface 31a of the first board 31 in the direction along the Z axis. The first board 31 and the solar cell 75 overlap each other in the direction along the Z axis. In other words, the solar cell 75 overlaps with the first board 31 in the thickness direction of the first board 31.

As described above, the protection portion 41 enclosing the solar cell 75 is transparent (translucent). In other words, the portion of the protection portion 41 which the light-receiving face 75a faces toward is translucent. The portion toward which the light-receiving face 75a faces is a portion overlapped with the light-receiving face 75a in a direction perpendicular to the light-receiving face 75a. In this manner, the protection portion 41 is an example of a first translucent portion.

The first wall 51 facing the light-receiving face 75a is also transparent (translucent). In other words, the portion of the cover 24 which the light-receiving face 75a faces toward is also translucent. The portion toward which the light-receiving face 75a faces is a portion overlapped with the light-receiving face 75a in a direction perpendicular to the light-receiving face 75a. In this manner, the first wall 51 is an example of a second translucent portion.

The solar cell 75 allows the light-receiving face 75a to receive light to generate electric power. External light such as sunlight is incident on the light-receiving face 75a through the translucent first wall 51 and the translucent protection portion 41. Therefore, the solar cell 75 generates the electric power.

The feeding cable 75c of the solar cell 75 is connected to, for example, the first board 31. The solar cell 75 supplies the generated electric power through the feeding cable 75c to the module 21. The module 21 can operate by the electric power generated by the solar cell 75 or can allow the battery 22 to be charged with the electric power generated by the solar cell 75.

In the electronic apparatus 12 according to the third embodiment, the solar cell 75 supplying the electric power to the module 21 is enclosed in the gel-like portion 23. The translucent protection portion 41 and the translucent first wall 51 which the light-receiving face 75a of the solar cell 75 faces toward are provided in the gel-like portion 23 and the cover 24, respectively. Therefore, by receiving the light passing through the protection portion 41 and the first wall 51, the electronic apparatus 12 can operate by the electric power generated by the solar cell 75, and the usage and available period of the electronic apparatus 12 are extended. Furthermore, the electric power generated by the solar cell 75 can be supplied to other device such as the device 11. Furthermore, the gel-like portion 23 encloses the solar cell 75, so that exposing the solar cell 75 to, for example, impact, a chemical agent, wind and rain, and a corrosion gas is suppressed. Since the electronic apparatus 12 can be easily adhered to the target face, it is possible to attach the solar cell 75 at a desired site.

In the third embodiment described above, the light passing through the translucent protection portion 41 and the translucent first wall 51 is incident on the light-receiving face 75a of the solar cell 75. But not limited thereto, for example, an opening exposing the light-receiving face 75a of the solar cell 75 to the outside may be provided in the protection portion 41 and the first wall 51. Therefore, since the light is directly incident on the light-receiving face 75a of the solar cell 75, the attenuation and diffusion of the light incident on the light-receiving face 75a is suppressed, so that the solar cell 75 can more efficiently generate the electric power.

### Fourth Embodiment

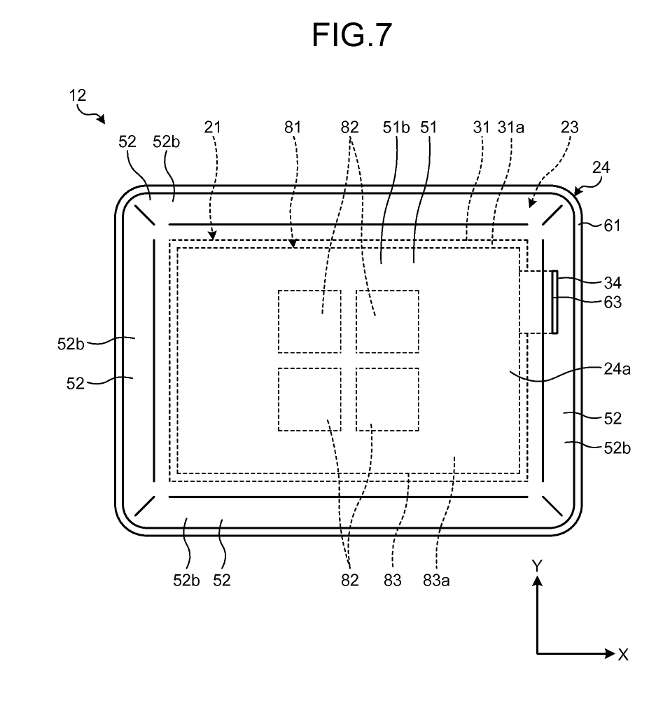

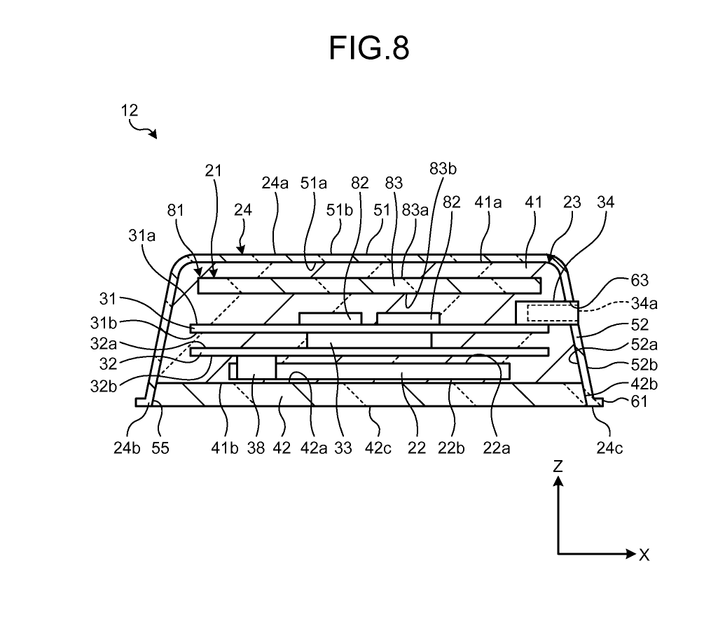

Hereinafter, a fourth embodiment will be described with reference to FIGS. 7 and 8. FIG. 7 is a plan view illustrating an electronic apparatus 12 according to the fourth embodiment. FIG. 8 is a cross-sectional view illustrating the electronic apparatus 12 according to the fourth embodiment.

As illustrated in FIG. 8, the module 21 according to the fourth embodiment further includes an illumination part 81. The illumination part 81 is an example of a light-emitting component and is also referred to as, for example, a light-transmitting component. The illumination part 81 is enclosed in the protection portion 41. The illumination part 81 includes a plurality of light sources 82 and a diffusing portion 83.

The light source 82 is, for example, an LED. In addition, the light source 82 may be the other light sources such as a fluorescent lamp. The light sources 82 are mounted on the first surface 31a of the first board 31. Therefore, the light sources 82 together with the first board 31 are enclosed in the protection portion 41. The light source 82 emits light, for example, in the direction where the first surface 31a faces.

The diffusing portion 83 is, for example, a silicon gel which is added with a diffusion material. In addition, the diffusing portion 83 may be the other members such as a diffusion plate. The diffusing portion 83 is formed in a substantially quadrangular plate shape. The diffusing portion 83 is enclosed in the protection portion 41 of the gel-like portion 23.

The diffusing portion 83 has a seventh surface 83a and an eighth surface 83b. The seventh surface 83a is a substantially even face facing in the direction along the Z axis. The seventh surface 83a faces in the direction where the light source 82 emits light. The eighth surface 83b is a substantially even face disposed at the opposite side of the seventh surface 83a. The eighth surface 83b faces in the direction opposite to the direction where the seventh surface 83a faces.

The seventh surface 83a of the diffusing portion 83 faces the first inner face 51a of the first wall 51 through the protection portion 41. The seventh surface 83a of the diffusing portion 83 is disposed at a position separated from the first inner face 51a of the first wall 51 in the direction along the Z axis. The first wall 51 and the diffusing portion 83 overlap each other in the direction along the Z axis. In other words, the diffusing portion 83 overlaps with the first wall 51 in the thickness direction of the first wall 51.

The eighth surface 83b of the diffusing portion 83 faces the first surface 31a of the first board 31 through the protection portion 41. Furthermore, the eighth surface 83b faces the light source 82 mounted on the first surface 31a. The eighth surface 83b of the diffusing portion 83 is disposed at a position separated from the first surface 31a of the first board 31 in the direction along the Z axis. The first board 31 and the diffusing portion 83 overlap each other in the direction along the Z axis. In other words, the diffusing portion 83 overlaps with the first board 31 in the thickness direction of the first board 31.

As described above, the protection portion 41 enclosing the illumination part 81 is transparent (translucent). In other words, the portion of the protection portion 41 disposed in the direction where the light source 82 emits the light is translucent. In this manner, the protection portion 41 is an example of a third translucent portion.

The first wall 51 facing the seventh surface 83a is also transparent (translucent). In other words, the portion of the cover 24 disposed in the direction where the light source 82 emits the light is also translucent. In this manner, the first wall 51 is an example of a fourth translucent portion.

The light emitted by the light source 82 passes through the protection portion 41 and is diffused by the diffusing portion 83. The light diffused by the diffusing portion 83 is emitted through the protection portion 41 and the first wall 51 of the cover 24 to the outside of the electronic apparatus 12. In this manner, the illumination part 81 emits the light to the outside of the electronic apparatus 12.

In the electronic apparatus 12 according to the fourth embodiment, the illumination part 81 emitting the light is enclosed in the gel-like portion 23. The translucent protection portion 41 and the translucent first wall 51 disposed in the direction where the illumination part 81 emits the light are provided in the gel-like portion 23 and the cover 24, respectively. Therefore, the illumination part 81 emits the light which is to pass through the translucent protection portion 41 and the translucent first wall 51. Accordingly, it is possible to illuminate the outside by using the illumination part 81. Furthermore, for example, in the case where the electronic apparatus 12 operates, the illumination part 81 is turned on, or in the case where the capacity of the battery 22 is small, the illumination unit is blinked. Therefore, by the light of the illumination part 81, the state of the electronic apparatus 12 can be notified to the outside. Since the electronic apparatus 12 can be easily adhered to the target face, it is possible to install the illumination part 81 at a desired site.

In the fourth embodiment, the light emitted by the illumination part 81 passes through the translucent protection portion 41 and the translucent first wall 51 to be emitted to the outside of the electronic apparatus 12. But not limited thereto, for example, an opening which exposes, for example, the diffusing portion 83 of the illumination part 81 to the outside may be provided in the protection portion 41 and the first wall 51. Therefore, since the light is directly emitted to the outside of the electronic apparatus 12, attenuation and diffusion of the light emitted by the illumination part 81 is suppressed, so that the illumination part 81 can more efficiently illuminate the outside of the electronic apparatus 12.

### Fifth Embodiment

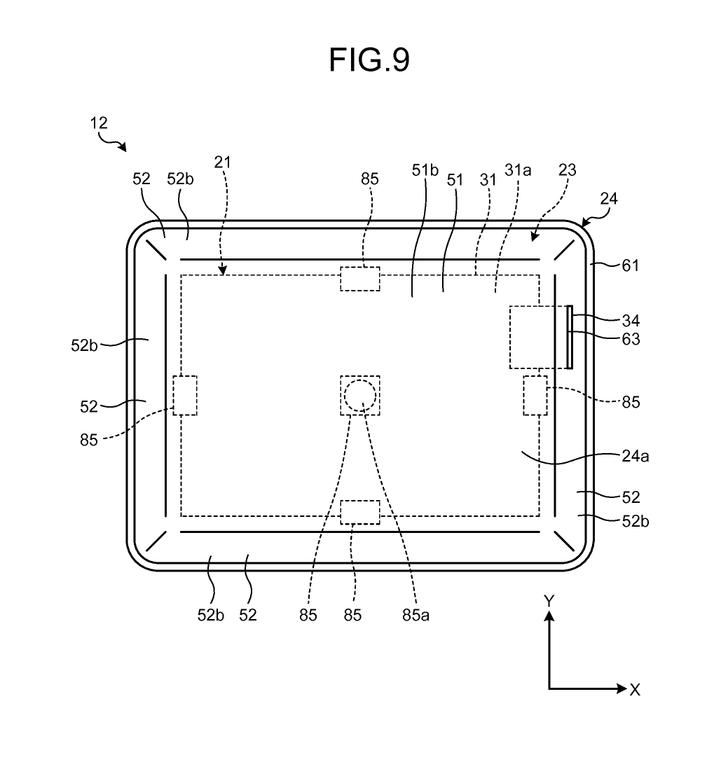

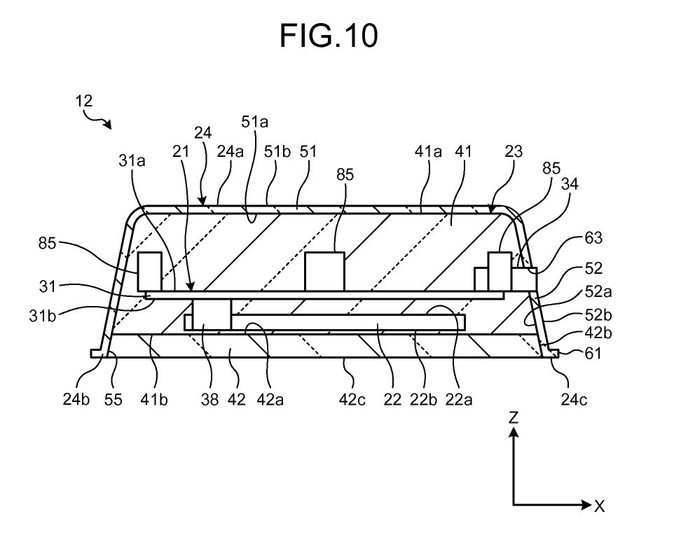

Hereinafter, a fifth embodiment will be described with reference to FIGS. 9 and 10. FIG. 9 is a plan view illustrating an electronic apparatus 12 according to the fifth embodiment. FIG. 10 is a cross-sectional view illustrating the electronic apparatus 12 according to the fifth embodiment.

As illustrated in FIG. 9, the module 21 according to the fifth embodiment further includes a plurality of cameras 85. The camera 85 is also referred to as, for example, an imaging portion. Each of the cameras 85 includes a lens 85a.

Each camera 85 is mounted on the first surface 31a of the first board 31. Therefore, the cameras 85 together with the first board 31 are enclosed in the protection portion 41. The lenses 85a of the cameras 85 direct in different directions. As illustrated in FIG. 9, the lens 85a of the one camera 85 is directed toward the first wall 51 of the cover 24, and the lenses 85a of the four cameras 85 direct toward the second walls 52.

As described above, the protection portion 41 enclosing the camera 85 is transparent (translucent). In other words, the portion of the protection portion 41 disposed in the direction where the lens 85a of the camera 85 is directed is translucent. The portion toward which the lens 85a is directed is a portion overlapped with the lens 85a in a direction perpendicular to the lens 85. In this manner, the protection portion 41 is an example of a fifth translucent portion.

The first and second walls 51 and 52 which the lenses 85a of the cameras 85 direct toward are also transparent (translucent). In other words, the portion of the cover 24 disposed in the direction where the lens 85a of the camera 85 is directed is translucent. The portion toward which the lens 85a is directed is a portion overlapped with the lens 85a in a direction perpendicular to the lens 85. In this manner, the first and second walls 51 and 52 are an example of a sixth translucent portion.

The camera 85 captures an image of the outside of the electronic apparatus 12 through the protection portion 41 and the first wall 51 or the second wall 52. The image captured by the camera 85 is transmitted, for example, from the first connector 34 through the external cable 13 to the device 11.

In the electronic apparatus 12 according to the fifth embodiment, the module 21 includes the camera 85. The translucent protection portion 41 and the translucent first and second walls 51 and 52 disposed in the direction where the camera 85 is directed are provided in the gel-like portion 23 and the cover 24, respectively. Therefore, the camera 85 captures the image of the outside through the translucent protection portion 41 and the translucent first and second walls 51 and 52. Since the electronic apparatus 12 can be easily adhered to the target face, it is possible to install the camera 85 at a desired site.

In the fifth embodiment, each camera 85 captures the image in the set one direction. However, the camera 85 is not limited thereto, but for example, by moving the direction where the lens 85a is directed, the direction where the image is to be captured may be changeable.

In the fifth embodiment, the camera 85 captures the image of the outside through the translucent protection portion 41 and the translucent first wall 51. But not limited thereto, for example, an opening which exposes the lens 85a of the camera 85 to the outside may be provided in the protection portion 41 and the first wall 51. Therefore, since the camera 85 can directly capture the image of the outside, the camera 85 can capture the clearer image.

### Sixth Embodiment

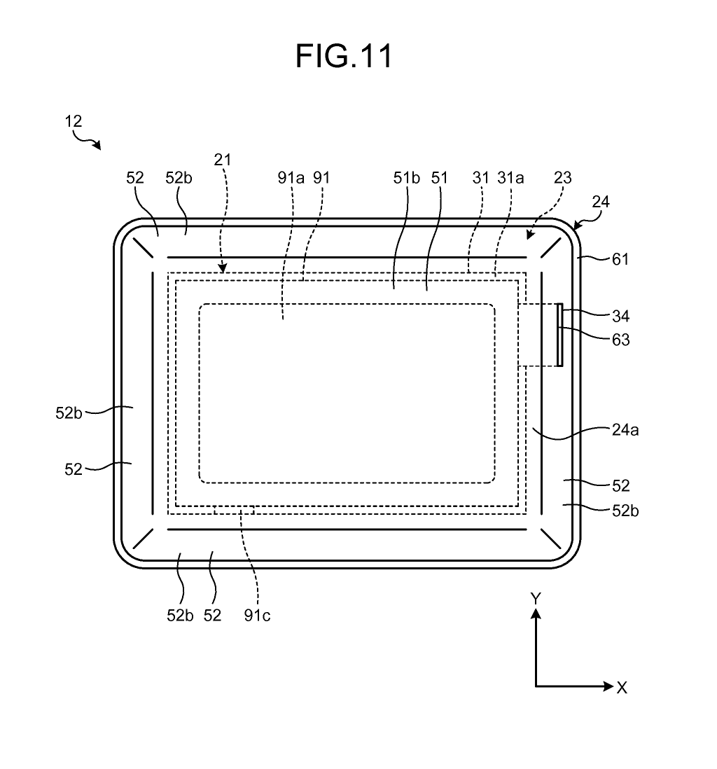

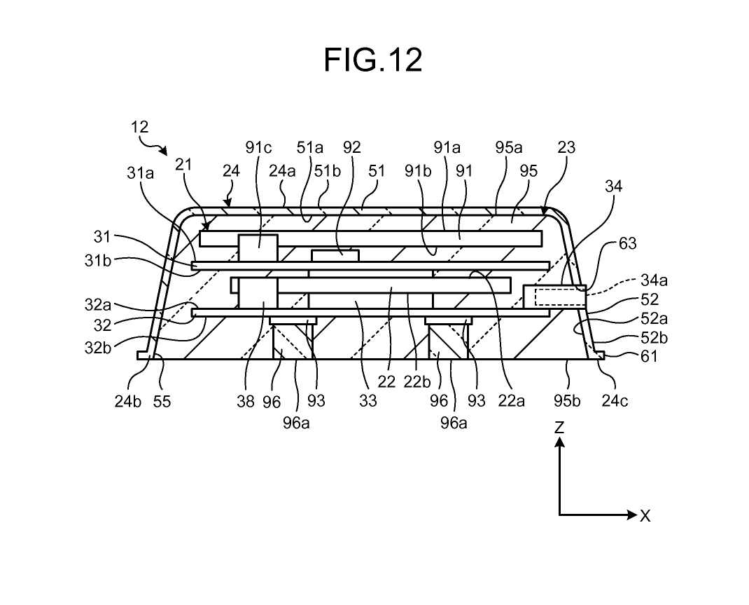

Hereinafter, a sixth embodiment will be described with reference to FIGS. 11 and 12. FIG. 11 is a plan view illustrating an electronic apparatus 12 according to the sixth embodiment. FIG. 12 is a cross-sectional view illustrating the electronic apparatus 12 according to the sixth embodiment.

As illustrated in FIG. 12, the module 21 according to the sixth embodiment includes a display part 91, an electrocardiographic sensor 92, and two electrodes 93. The electrocardiographic sensor 92 is also referred to as, for example, a biosensor, a sensor, and a part. The electrode 93 is also referred to as, for example, a sensing portion, a conductive portion, and a portion.

The electrocardiographic sensor 92 is mounted on, for example, the first surface 31a of the first board 31. In addition, the electrocardiographic sensor 92 may be mounted on the other sites such as the second surface 31b, the third surface 32a, and the fourth surface 32b.

The electrode 93 is an electrode used for the electrocardiographic sensor 92 to sense a cardiac action potential and is connected to the electrocardiographic sensor 92. In addition, the electrode 93 is not limited thereto. The electrode 93 is mounted on the fourth surface 32b of the second board 32. In addition, the electrode 93 may be mounted at other sites. The electrode 93 faces in the direction where the fourth surface 32b faces.

The battery 22 according to the sixth embodiment is disposed, for example, between the first board 31 and the second board 32 in the direction along the Z axis. In addition, the battery 22 may be disposed as the other positions. The battery 22 is disposed to a position other than the position toward which the electrode 93 faces.

The gel-like portion 23 according to the sixth embodiment has an insulating portion 95 and two conductive portions 96. The insulating portion 95 and the conductive portions 96 are a sticky gel-like portion having transparency (translucency). The insulating portion 95 is made of, for example, an insulating silicon gel. The conductive portion 96 is made of, for example, a silicon gel which is added with a conductive material, so that the conductive portion 96 has conductivity.

The insulating portion 95 encloses the module 21 and the battery 22. In other words, the insulating portion 95 covers the module 21 and the battery 22. Therefore, similarly to the protection portion 41 according to the first embodiment, the insulating portion 95 is attached to each surface of the components of the module 21 and the battery 22.

A portion of the insulating portion 95 is interposed among the first board 31, the second board 32, and the battery 22. Therefore, the insulating portion 95 maintains the first board 31, the second board 32, and the battery 22 to be at positions separated from each other.

The insulating portion 95 has a sixth adhesive face 95a and a seventh adhesive face 95b. The seventh adhesive face 95b is an example of a first face according to the sixth embodiment and is also referred to as, for example, a sticky portion, an attaching portion, and a viscous portion.

The sixth adhesive face 95a is an outer face of the insulating portion 95 facing the cover 24. The sixth adhesive face 95a is adhered to the first inner face 51a of the first wall 51 and the second inner face 52a of the second wall 52.

Similarly to the fifth adhesive face 42c according to the first embodiment, the seventh adhesive face 95b is a substantially even face facing in the direction along the Z axis. The seventh adhesive face 95b is exposed to the outside of the electronic apparatus 12 by the first opening portion 55.

The conductive portion 96 has an eighth adhesive face 96a. Similarly to the seventh adhesive face 95b, the eighth adhesive face 96a is an example of a first face according to the sixth embodiment and is also referred to as, for example, a sticky portion, an attaching portion, or a viscous portion.

The conductive portion 96 extends from the corresponding electrode 93 in the direction along the Z axis. The eighth adhesive face 96a of the conductive portion 96 forms substantially the same plane as the seventh adhesive face 95b of the insulating portion 95. Namely, the seventh adhesive face 95b of the insulating portion 95 and the eighth adhesive face 96a of the conductive portion 96, which form an integrated face, are exposed by the first opening portion 55.

The display part 91 is formed in a substantially quadrangular plate shape. The display part 91 is enclosed in the insulating portion 95 of the gel-like portion 23. The display part 91 has a display face 91a, a back face 91b, and a connection cable 91c. The display face 91a is an example of a third face. The display face 91a is a substantially even face facing in the direction along the Z axis. The back face 91b is a substantially even face disposed at the opposite side of the display face 91a. The back face 91b faces in the direction opposite to the direction where the display face 91a faces.

The display face 91a of the display part 91 faces the first inner face 51a of the first wall 51 through the insulating portion 95. The display face 91a of the display part 91 is disposed at a position separated from the first inner face 51a of the first wall 51 in the direction along the Z axis. The first wall 51 and the display part 91 overlap each other in the direction along the Z axis. In other words, display part 91 overlaps with the first wall 51 in the thickness direction of the first wall 51.

The back face 91b of the display part 91 faces the first surface 31a of the first board 31 through the insulating portion 95. The back face 91b of the display part 91 is disposed at a position separated from the first surface 31a of the first board 31 in the direction along the Z axis. The first board 31 and the display part 91 overlap each other in the direction along the Z axis. In other words, display part 91 overlaps with the first board 31 in the thickness direction of the first board 31.

As described above, the insulating portion 95 enclosing the display part 91 is transparent (translucent). In other words, the portion of the insulating portion 95 which the display face 91a faces toward is translucent. The portion toward which the display face 91a faces is a portion overlapped with the display face 91a in a direction perpendicular to the display face 91a. In this manner, the insulating portion 95 is an example of a seventh translucent portion.

The first wall 51 facing the display face 91a is also transparent (translucent). In other words, the portion of the cover 24 where the display face 91a faces toward is translucent. The portion toward which the display face 91a faces is a portion overlapped with the display face 91a in a direction perpendicular to the display face 91a. In this manner, the first wall 51 is an example of an eighth translucent portion.

The display part 91 is connected to, for example, the first board 31 by the connection cable 91c. The display part 91 is controlled through the connection cable 91c in other units of the module 21 such as the first board 31 to display the image on the display face 91a. For example, the display part 91 displays a sensing result of the electrocardiographic sensor 92 on the display face 91a.

The electronic apparatus 12 formed as described above is used as, for example, an electrocardiographic monitor device as described hereinafter. In addition, the electronic apparatus 12 is not limited to the usage described below, but the electronic apparatus may be used for other usages.

The seventh adhesive face 95b of the insulating portion 95 and the eighth adhesive face 96a of the conductive portion 96 which are exposed by the first opening portion 55 to form an integrated face are brought into contact with a human body. Therefore, the seventh adhesive face 95b of the insulating portion 95 and the eighth adhesive face 96a of the conductive portion 96 which are a sticky gel-like portion are stuck with the human body.

The electrode 93 is electrically connected to the human body through the corresponding conductive portion 96. In other words, the electrode 93 is electrically connected to the portion (human body) which is in contact with the seventh adhesive face 95b of the insulating portion 95 and the eighth adhesive face 96a of the conductive portion 96 which form the integrated face. The electrocardiographic sensor 92 senses the cardiac action potential of the human body from the electrode 93 which is electrically connected to the human body through the conductive portion 96.

The seventh adhesive face 95b of the insulating portion 95 and the eighth adhesive face 96a of the conductive portion 96 can be peeled off from the human body. The seventh and eighth contact faces 95b and 96a peeled off from the human body continue to have stickiness. Therefore, the seventh and eighth contact faces 95b and 96a can be adhered to the target face such as the human body again. In this manner, since the electronic apparatus 12 can be repetitively adhered to/peeled off from the target face, it is possible to easily adjust the contact position.

For example, in the case where the electronic apparatus 12 is repetitively adhered to/peeled off from the human body, the adhesive forces of the seventh and eighth contact faces 95b and 96a may be decreased due to sebum or dust. In this case, the seventh and eighth contact faces 95b and 96a recover the adhesive forces, for example, by cleaning with water and detergent and drying. Accordingly, a decrease in the adhesive force of the electronic apparatus 12 or a deterioration in sanitary condition is suppressed.

The insulating portion 95 and the conductive portion 96 described above are manufactured, for example, as described hereinafter. In addition, not limited to the method described hereinafter, the insulating portion 95 and the conductive portion 96 may be manufactured according to the other methods.

In the state that the module 21 is accommodated inside the first opening portion 55 of the cover 24, masking blocks are disposed in the portions where the conductive portions 96 are formed. Next, the material of the insulating portion 95 having fluidity is supplied to the first opening portion 55 of the cover 24. The material is hardened by heating. Therefore, the insulating portion 95 is formed.

Next, by removing the masking blocks, cavities are formed in the portions where the conductive portions 96 are to be formed. The material of the conductive portion 96 having fluidity is supplied to the cavities. The material is hardened by heating. Therefore, the conductive portions 96 are formed.

According to the processes heretofore, the electronic apparatus 12 is manufactured. In addition, the material of the insulating portion 95 and the material of the conductive portion 96 may be simultaneously heated. In addition, in the case where the electronic apparatus 12 is adhered to the human body as described above, the adhesive force of the silicon gel which is the material of the gel-like portion 23 may be set to be lower than the adhesive force of the silicon gel in the case where the electronic apparatus 12 is adhered to the target face other than the human body.

In the electronic apparatus 12 according to the sixth embodiment, the module 21 includes the display part 91. The translucent insulating portion 95 and the translucent first wall 51 disposed in the direction where the display face 91a of the display part 91 faces are provided in the gel-like portion 23 and the cover 24, respectively. Therefore, the image displayed on the display face 91a can be visually recognized through the translucent insulating portion 95 and the translucent first wall 51. Since the electronic apparatus 12 can be easily adhered to the target face, it is possible to easily install the display part 91 at a desired site.

The module 21 includes the electrode 93 configured so as to be electrically connected to the portion which is in contact with the seventh adhesive face 95b of the insulating portion 95 and the eighth adhesive face 96a of the conductive portion 96 which form the integrated face. Therefore, the seventh and eighth adhesive faces 95b and 96a are adhered to the portion (for example, human body) which is connected to the electrode 93, so that the portion and the electrode 93 are electrically connected to each other. Accordingly, it is possible to easily connect the electrode 93 to the portion.

In addition, in the sixth embodiment, the electrode 93 is electrically connected to the human body through the conductive portion 96, but the electrode 93 is not limited thereto. For example, the electrode 93 may be exposed from the seventh adhesive face 95b of the insulating portion 95. Therefore, in the case where the seventh adhesive face 95b is adhered to the human body, since the electrode 93 is in contact with the human body, the electrode 93 is electrically connected to the human body.

The module 21 includes the electrocardiographic sensor 92 which is connected to the electrode 93. Therefore, the seventh and eighth adhesive faces 95b and 96a are adhered to the human body, so that the electrocardiographic sensor 92 can sense the cardiac action potential by using the electrode 93. Accordingly, for example, without another member such as a double-sided tape or a sucker, the electrode 93 of the electrocardiographic sensor 92 can be electrically connected to the human body.

In the sixth embodiment, the image displayed on the display face 91a of the display part 91 can be visually recognized through the translucent protection portion 41 and the translucent first wall 51. But not limited thereto, for example, an opening exposing the display face 91a of the display part 91 may be provided in the protection portion 41 and the first wall 51. Therefore, the image displayed on the display face 91a is directly visually recognized, so that the image displayed on the display face 91a can be easily recognized.

### Seventh Embodiment

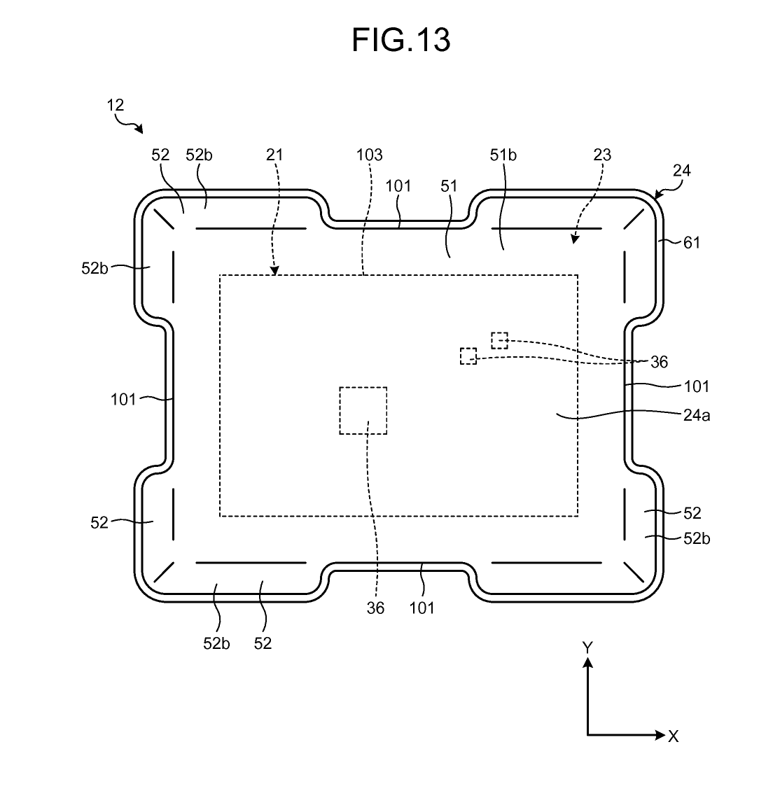

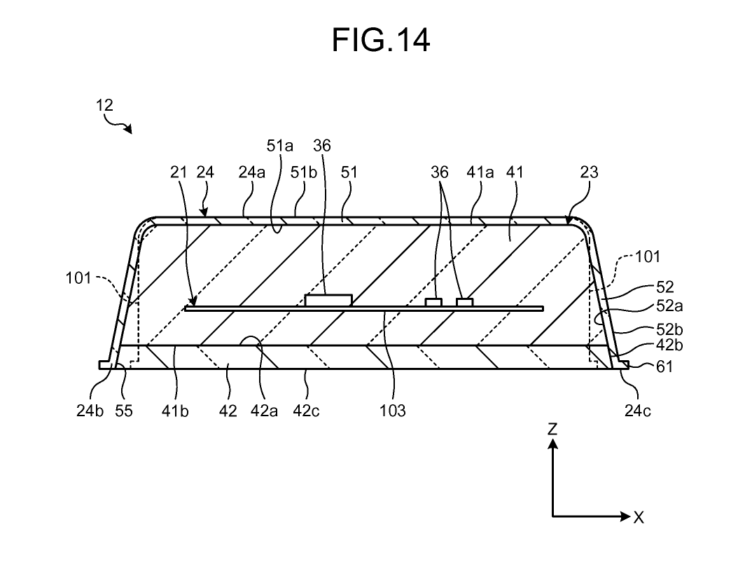

Hereinafter, a seventh embodiment will be described with reference to FIGS. 13 and 14. FIG. 13 is a plan view illustrating an electronic apparatus 12 according to the seventh embodiment. FIG. 14 is a cross-sectional view illustrating the electronic apparatus 12 according to the seventh embodiment.

As illustrated in FIG. 13, four held portions 101 are provided on the second walls 52 of the cover 24 according to the seventh embodiment. The held portion 101 is an example of at least one of a recess and a projection, and also referred to as, for example, a concave portion, a recess, a cut-off, and a grip portion.

In this embodiment, the held portion 101 is a portion which is recessed from the second wall 52. In other words, the held portion 101 is a portion, in the vicinity of which protrusions are provided. The held portion 101 extends from the first end portion 24a of the cover 24 in the direction toward the second end portion 24b. For example, a width of the held portion 101 is set to be larger than a width of user's finger which treats electronic apparatus 12.

As illustrated in FIG. 14, the module 21 according to the seventh embodiment includes an FPC 103. The FPC 103 is an example of a board that is flexible. For example, the plurality of the electronic components 36 are mounted on the FPC 103. The FPC 103 is more flexible than the electronic components 36.

In the electronic apparatus 12 according to the seventh embodiment, the module 21 includes the FPC 103. Although the gel-like portion 23 is deformed by an external force, the FPC 103 is deformed according to the deformation of the gel-like portion 23. Therefore, damage of the module 21 is suppressed.

The cover 24 includes the second walls 52 which extend in the direction intersecting the fifth adhesive face 42c and where the held portion 101 is provided. Therefore, for example, a user can grasp the electronic apparatus 12 by hitting the held portion 101 with a finger, so that it is possible to easily attach the electronic apparatus 12 to the target face.

In the seventh embodiment, the held portion 101 is a portion recessed from the second wall 52. But not limited thereto, the held portion 101 may be other easily-held portions such as a handle, a protrusion, a slit, and a surface-processed portion. Furthermore, in the seventh embodiment, the four held portions 101 are provided on the second wall 52, but the number of held portions 101 is not limited thereto.

### Eighth Embodiment

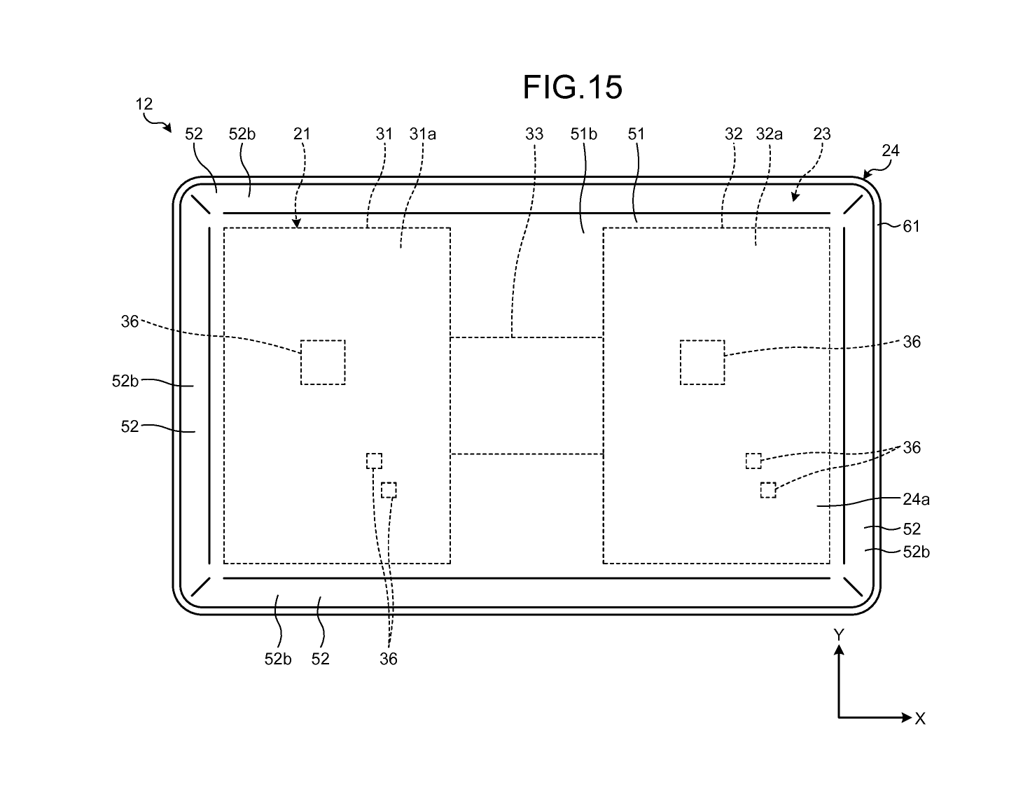

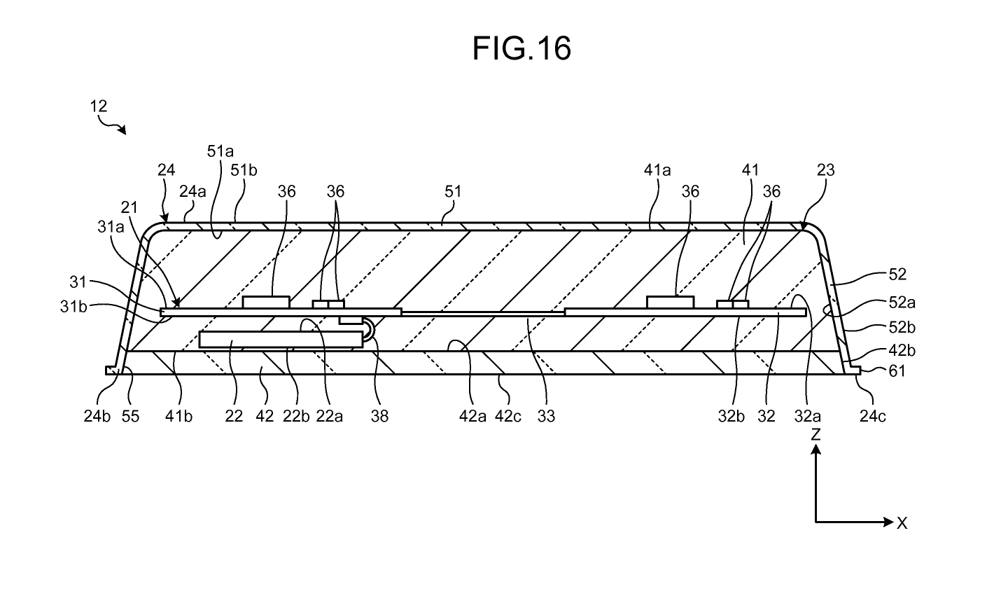

Hereinafter, an eighth embodiment will be described with reference to FIGS. 15 and 16. FIG. 15 is a plan view illustrating an electronic apparatus 12 according to the eighth embodiment. FIG. 16 is a cross-sectional view illustrating the electronic apparatus 12 according to the eighth embodiment.

As illustrated in FIG. 15, the electronic apparatus 12 according to the eighth embodiment extends in the direction along the X axis. The direction along the X axis is an example of a first direction. Therefore, the gel-like portion 23 and the cover 24 also extend in the direction along the X axis.

The first to third boards 31 to 33 are arranged in the direction along the X axis. The second board 32 is disposed to be separated from the first board 31 in the direction along the X axis. The third board 33 extends in the direction along the X axis and connects the first board 31 and the second board 32. Therefore, the third board 33 is disposed between the first board 31 and the second board 32 in the direction along the X axis.

The third board 33 is disposed in the central portion of the gel-like portion 23 in the direction along the X axis. In other words, at least a portion of the third board 33 is disposed at the center between the one end portion and the other end portion of the gel-like portion 23 in the direction along the X axis.

In the electronic apparatus 12 according to the eighth embodiment, the third board 33 is disposed between the first board 31 and the second board 32 in the direction along the X axis where the gel-like portion 23 extends. Furthermore, the third board 33 is disposed in the central portion of the gel-like portion 23 in the direction along the X axis which is easily bent and where stress concentration easily occurs when an external force is exerted on the gel-like portion 23. Therefore, although the gel-like portion 23 is bent due to the external force, the third board 33 absorbs the external force, for example, damage of the first and second boards 31 and 32 or the solder connection portions of the components mounted in the first and second boards 31 and 32 is suppressed.

According to at least one of the embodiments described heretofore, the sticky gel-like portion encloses the units. At least a portion of the surface of the gel-like portion is exposed to the outside. Therefore, the electronic apparatus is adhered to the target face without other members such as a double-sided tape.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. An electronic apparatus comprising:
a board;
a cover configured to be provided with a first opening, the cover being flexible;
a first gel-like member that is accommodated in the cover, the first gel-like member being sticky and comprising a first face exposed by the first opening; and
a second gel-like member accommodated in the cover, the second gel-like member being sticky and conductive, the second gel-like member enclosing the board.

2. The electronic apparatus according to claim 1, further comprising a battery that is enclosed in the second gel-like member and that supplies electric power to the board.

3. The electronic apparatus according to claim 1, wherein the second gel-like member is added with a reinforcement material.

4. The electronic apparatus according to claim 1, wherein the second gel-like member has thermal conductivity higher than that of the first gel-like member.

5. The electronic apparatus according to claim 1 further comprising an antenna that is disposed in the second gel-like member.

6. The electronic apparatus according to claim 1, wherein the board comprises a first board, a second board and a third board, the third board connecting the first board and the second board, the third board being more flexible than the first board and being more flexible than the second board.

7. The electronic apparatus according to claim 6, wherein the first board and the second board overlap each other in a thickness direction of the first board.

8. The electronic apparatus according to claim 6, wherein
each of the first and second gel-like members extends in a first direction, and
the third board is disposed between the first board and the second board in the first direction and is disposed at a central portion of the second gel-like member in the first direction.

9. The electronic apparatus according to claim 1, wherein the board is flexible.

10. The electronic apparatus according to claim 1, wherein the cover comprises an end and a protrusion, the first opening provided at the end, the protrusion protruding in a direction along the first face toward an outside of the first opening from the end.

11. The electronic apparatus according to claim 1, wherein the cover comprises a wall, the wall extending in a second direction intersecting the first face and provided with at least one of a recess and a projection.

12. The electronic apparatus according to claim 1 further comprising a connector that is disposed in the second gel-like member, the connector provided with a second opening, wherein
the cover is provided with a third opening exposed by the second opening.

13. The electronic apparatus according to claim 1, further comprising a solar cell enclosed in the second gel-like member, the solar cell comprising a second face, configured to generate electric power by receiving light through the second face and configured to supply the electric power to the board, wherein
the second gel-like member comprises a first translucent portion toward which the second face faces, and
the cover comprises a second translucent portion toward which the second face faces.

14. The electronic apparatus according to claim 1 further comprising a light-emitting component enclosed in the second gel-like member, the light-emitting component configured to emit light, wherein
the second gel-like member comprises a third translucent portion disposed in a direction where the light-emitting component configured to emit the light, and
the cover comprises a fourth translucent portion disposed in the direction where the light-emitting component emits the light.

15. The electronic apparatus according to claim 1 further comprising a camera enclosed in the second gel-like member, wherein
the second gel-like member comprises a fifth translucent portion disposed in a direction where the camera is directed, and
the cover comprises a sixth translucent portion disposed in the direction where the camera is directed.

16. The electronic apparatus according to claim 1 further comprising a displaying component enclosed in the second gel-like member, the displaying component comprising a third face and configured to display an image on the third face, wherein
the second gel-like member comprises a seventh translucent portion toward which the third face faces, and
the cover comprises an eighth translucent portion toward which the third face faces.

17. An electronic apparatus comprising:
a component; and
a sticky gel-like portion enclosing the component and comprises a surface, at least a portion of the surface configured to be exposed to an outside.

18. An electronic apparatus comprising:
a component;
a cover that is provided with a first opening; and
a sticky gel-like portion enclosing the component, accommodated in the cover, and comprising a first face configured to be exposed by the first opening.

19. The electronic apparatus according to claim 18, wherein the component comprises an electrode configured to be electrically connected to a portion being in contact with the first face.

20. The electronic apparatus according to claim 19, wherein the component comprises an electrocardiographic sensor connected to the electrode.
